(19) United States
(12) Patent Application Publication
(10) Pub. No.: US 2009/0021695 A1
(43) Pub. Date: Jan. 22, 2009

(54) Display of Ocular Movement

(76) Inventor: Frank Scarpino, Centerville, OH (US)

Correspondence Address: WITHERS & KEYS, LLC, P. O. BOX 71355, MARIETTA, GA, 30007-1355, US

(21) Appl. No.: 11/911,192

(22) PCT Filed: Apr. 11, 2006
(86) PCT No.: PCT/US2006/013511

§ 371 Date: Aug. 1, 2008

Related U.S. Application Data
(60) Provisional application No. 60/670,084, filed on Apr. 11, 2005.
(60) Provisional application No. 60/719,523, filed on Sep. 22, 2005.

(51) Int. Cl. A61B 3/113

(52) U.S. Cl. 351/210; 351/246

(57) ABSTRACT

Ocular movement of a subject is displayed in one or more windows of a user interface allowing a technician and/or clinician to observe the ocular movement such as to properly administer various tests for visual, vestibular, and neurological disorders as well as for diagnosing such disorders. When displaying the ocular movement, the video of the ocular movement being displayed may be panned to adjust the position of each eye within a display window as desired, such as to center the pupils and to provide a common horizontal location for both left and right pupils. Additionally, zooming in or out on the video of ocular movement may be provided to allow artifacts of the video stream to be effectively cropped from the display window and to allow the details of the ocular movement to be adequately visible. Furthermore, the display window size may be increased such that the details of the ocular movement are enlarged to allow the clinician and/or technician to better see those details even from a distance.

### RELATED APPLICATIONS

The present application claims priority to U.S. Provisional Application 60/670,084, filed on Apr. 11, 2005, and entitled BALANCE AND VESTIBULAR DISORDER DIAGNOSIS AND REHABILITATION, which is incorporated herein by reference. The present application also claims priority to U.S. Provisional Application 60/719,523, filed on Sep. 22, 2005, and entitled BALANCE AND VESTIBULAR DISORDER DIAGNOSIS AND REHABILITATION, which is also incorporated herein by reference.

### TECHNICAL FIELD

The present application is directed to the display of ocular movement. More particularly, the present application is directed to the display of ocular movement by manipulating aspects of the display.

### BACKGROUND

Ocular movement is observed by clinicians in order to diagnose various medical disorders including visual, vestibular, and/or neurological problems that the subject may be experiencing. The subject is asked to view a visual display that provides a stimulus to the subject. The stimulus may be voluntary, in that the subject chooses to visually respond to the stimulus, or the stimulus may be involuntary in that the eyes of the subject involuntarily respond to the stimulus. The ocular movement resulting from the stimulus is revealing to the clinician.

In order to assist the clinician in diagnosing the problem being experienced by the subject, the ocular movement may be captured on video and displayed within a graphical user interface of a computer application. The computer application may make measurements of the ocular movement of each eye which can be graphed and analyzed. The display of the video of the ocular movement assists the technician running the test by allowing the technician to make sure that the eyes are being properly tracked by the computer application. Furthermore, the display of the video of the ocular movement assists the physician by allowing the physician to see the ocular movement without directly staring at the patient while the patient is observing and responding to the stimulus. Furthermore, this video may be recorded for future playback by the physician.

To capture this video, goggles having cameras for each eye are placed onto the subject. The cameras capture the video footage of the ocular movement of each eye and provide the video stream to the computer application so that the ocular movement can be displayed and tracked. However, for the ocular movement to be properly obtained, the goggles must be properly located on the face of the subject so that each eye is being adequately recorded. This requires that the technician administering the test must spend lengthy amounts of time properly adjusting the goggles to get the best video capture.

This need for adjustment of the goggles presents many problems. Because one subject has facial features that may vary drastically from another, the amount of physical adjustment to the goggles may not provide ideal video capture of the ocular movement since the adjustment may fail to properly center the eyes within the video frames being captured. Additionally, the size of the eyes within the video frame may be inadequate for proper tracking and/or viewing. Furthermore, the subject may be having the ocular movement test performed due to a balance or dizziness disorder such that moving the head of the subject while attempting to physically adjust the goggles positioning may be uncomfortable or even unbearable.

### SUMMARY

Embodiments of the present invention address these issues and others by providing control of the display of the ocular movement via the user interface being used to display the ocular movement. Such control may include panning of the video being displayed in order to change the position of the eyes within the video window, such as to center each eye on the horizontal and vertical axes. Such control may additionally or alternatively include zooming in or out of the video being displayed, such as to zoom in to make the pupil larger for proper tracking and/or to zoom in to eliminate artifacts such as parts of the goggles that may be captured by the cameras. Such control may additionally or alternatively include enlarging the video window to increase the size on the display screen of the video of ocular movement being shown, such as to allow the technician or clinician to move some distance from the display screen and continue to see the ocular movement.

One embodiment involves obtaining a sequence of digitized video frames of the ocular movement at a first resolution. A portion of each frame of the sequence of digitized video frames of the ocular movement is displayed, the portion being at a second resolution lower than the first resolution and being displayed at a first display resolution.

A first user input is received while displaying in sequence the portion of each frame, and in response to the received first user input, the portion is panned within the subsequent frames of the ocular movement being displayed.

Another embodiment is a computer system for displaying ocular movement. The computer system includes a first input receiving a sequence of digitized video frames of the ocular movement at a first resolution and a memory storing at least a portion of each digitized video frame being received. The computer system also includes a second input receiving a first user input and a processor that initiates displaying in sequence a portion of each frame of the sequence of digitized video frames of the ocular movement. The portion is at a second resolution lower than the first resolution and is displayed at a first display resolution, and in response to the received first user input the processor initiates panning the portion within the subsequent frames of the ocular movement being displayed.

Another embodiment is a computer readable medium having instructions encoded thereon that perform acts that include obtaining a sequence of digitized video frames of the ocular movement at a first resolution. The acts further include displaying in sequence a portion of each frame of the sequence of digitized video frames of the ocular movement, the portion being at a second resolution lower than the first resolution and being displayed at a first display resolution. Additionally, the acts include receiving a first user input while displaying in sequence the portion of each frame, and in response to the received first user input panning the portion within the subsequent frames of the ocular movement.

### DESCRIPTION OF THE DRAWINGS

FIG. 1 shows an example of an operating environment for the various embodiments for displaying ocular movement, including goggles and a computer running a testing application.

FIG. 2 shows an example of the computer running the testing application to generate the display of ocular movement according to an embodiment.

FIG. 3 shows one example of the relationship of video capture and display processing modules and operations according to an embodiment.

FIG. 4 shows one example of the operational flow performed by the testing application when controlling the display of ocular movement according to an embodiment.

FIG. 5-D show the various resolutions of the video frames used to display the ocular movement according to one illustrative embodiment.

FIG. 6 shows a screenshot of an instant where one frame for a right eye and a left eye is being displayed and where the right eye and the left eye are at full frame.

FIG. 7 shows a screenshot of an instant where one frame for the right eye and one frame for the left eye have been zoomed to a portion of full frame.

FIG. 8 shows a screenshot of an instant where one frame for the right eye has been panned horizontally from the frame shown in FIG. 7.

FIG. 9 shows a screenshot of an instant where one frame for the left eye has been panned horizontally from the frame shown in FIG. 7.

FIG. 10 shows a screenshot of an instant where one frame for the right eye has been panned vertically from the frame shown in FIG. 8.

FIG. 11 shows a screenshot of an instant where one frame for the left eye has been panned vertically from the frame shown in FIG. 8.

FIG. 12 shows a screenshot of an instant where one frame for the right eye and one frame for the left eye have been magnified to an increased display resolution.

### DETAILED DESCRIPTION

Various embodiments are disclosed herein for displaying ocular movement. According to illustrative embodiments disclosed herein, the display of a sequence of video frames of the ocular movement allows for panning of the position of the right and/or left eye within display windows. According to various embodiments, the display of the sequence of video frames allows for zooming in or out on the video frames of ocular movement and/or increasing the display resolution of the video frames thereby making them visible from a distance.

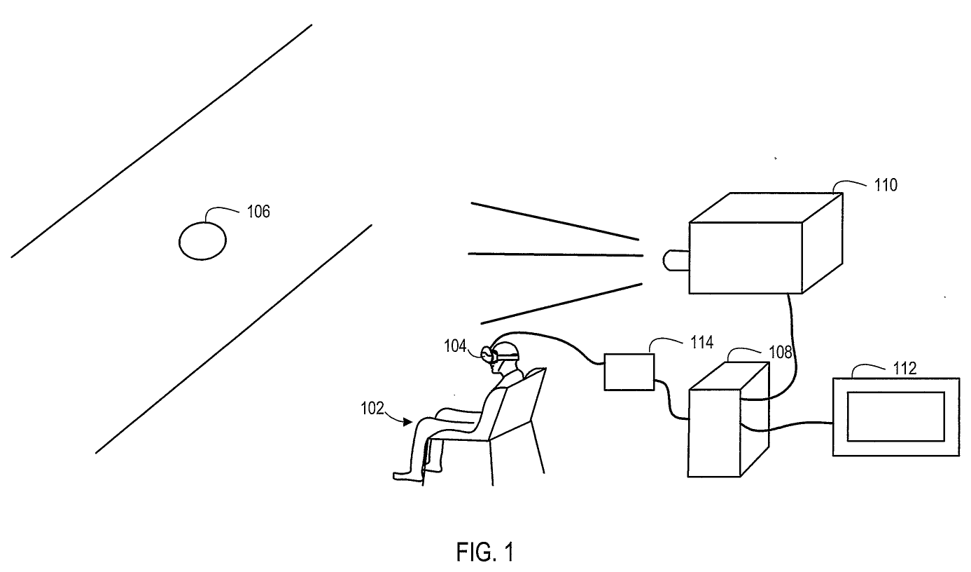

FIG. 1 shows one example of an operating environment where ocular movement is displayed in accordance with the illustrative embodiments. In this example, a subject 102 is wearing goggles 104 that have video capture ability. For example, the goggles may shine infrared light toward each eye and a separate infrared camera for each eye records the infrared video of the ocular movement. It will be appreciated that various other manners of initially generating the video signal are possible, such as using tri-pod mounted cameras, using visible light cameras as opposed to infrared cameras, and so forth.

In this example, the goggles 104 feeds a video signal to a control box 114 which powers the cameras and infrared emitters of the goggles 104 and then outputs the video signal, e.g., an NTSC signal, to a computer 108. The control box 114 may pass through the video signal to the computer 108 or may digitize the video signal, compress the digitized video signal, and so forth prior to sending the digitized video signal to the computer 108.

The computer 108 may employ video signal capture techniques to digitize, compress, and otherwise process the video signal where the control box 114 passes the video signal. Where the control box 114 has already digitized the video signal, the computer 108 may compress the digitized video signal if necessary and may perform additional video processing techniques. The computer 108 may store the digitized video signal for subsequent playback and/or for transport.

The computer 108 may also display the video, either in substantially real-time as the video of the ocular movement is being captured or after some delay, on a video screen 112. A technician or clinician may view the ocular movement on the display screen 112 and may manipulate the display of the ocular movement in accordance with the various embodiments disclosed herein by interacting with user input devices of the computer 108.

The computer 108 may also generate a stimulus display that is then shown to the subject 102. In the example shown, the stimulus display signal is provided to a projector 110 which then projects the stimulus display so that it is visible by the subject 104. In this particular example, the stimulus is a dot 106 that the subject 102 may stare at. The dot may move so that the subject 102 must move his or her eyes to follow the movement of the dot 106. It will be appreciated that the stimulus may be of various forms, such as optokinetic stimuli, saccades, smooth pursuit, and the like. It will also be appreciated that other manners of displaying the stimulus are available, including placing a video display device such as a liquid crystal display, plasma display, and the like in front of the subject 102 rather than projecting the image onto a wall or screen.

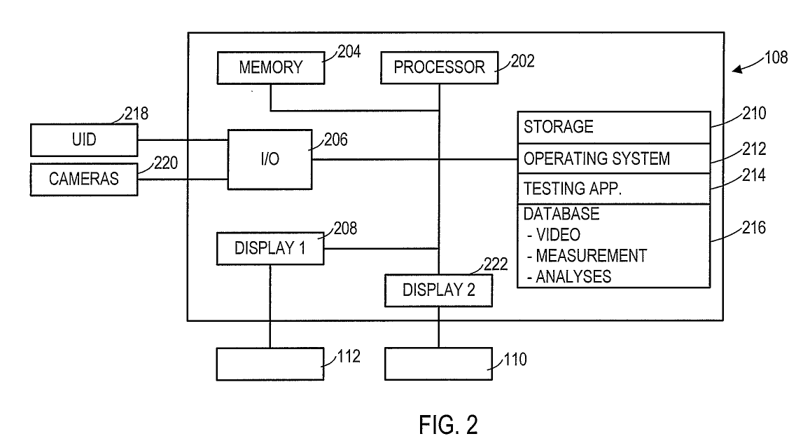

FIG. 2 shows one example of the computer 108. This computer 108 includes a processor 202, memory 204, input/output (I/O) 206, mass storage 210, a first display adapter 208 and a second display adapter 222. The processor 202 may be a general purpose programmable processor, an application specific processor, hardwired digital logic, and so forth. The memory 204 may include volatile and non-volatile memory, may be separate from the processor 202 or may be integrated with the processor 202. For embodiments where the computer 108 is performing various tasks such as real-time tracking and analysis in addition to displaying the ocular movement, a dual core processor implementing simultaneous parallel threads may be desirable to prevent reduction in speed of the display of the ocular movement.

The mass storage 210 is accessed by the processor through a data bus 201. Examples of the mass storage 210 include magnetic drives and/or optical drives. The mass storage 210 may store an operating system 212, a testing application 214, and a database 216. The processor 202 may access the operating system 212 to perform basic tasks and to execute the testing application 214.

The testing application 214 provides logical operations performed by the processor 202 to obtain the video frames of the ocular movement and to initiate the display of the ocular movement via one of the display adapters. The testing application provides for manipulation of the display of the ocular movement, such as panning, zooming, and magnification. Additionally, the testing application may provide logical operations performed by the processor 202 to initiate the display of the stimulus via one of the display adapters and to record the video of the ocular movement. The testing application may provide many other features as well, such as but not limited to tracking the movement of the pupils, recording the data points representing the movement and displaying the movement in a graph, analyzing the movement in relation to set criteria, and displaying charts that are representative of the analyses.

The testing application 214 may also maintain a database 216 of test data for each subject. The test data may include the digitized and compresses video sequences, the measured data points, and the analyses. The database 216 may be used to revisit the testing, including the video, data points, and analyses at some later time after the initial testing has been completed. Furthermore, the database entries may be transportable to computer systems at remote locations.

The processor 202, the memory 204, and storage 210 each in their various forms represent examples of computer readable media. Computer readable media contain instructions for performing the logical operations of the various embodiments. Computer readable media include storage media, such as electronic, magnetic, and optical storage, as well as communications media such as wired and wireless data connections.

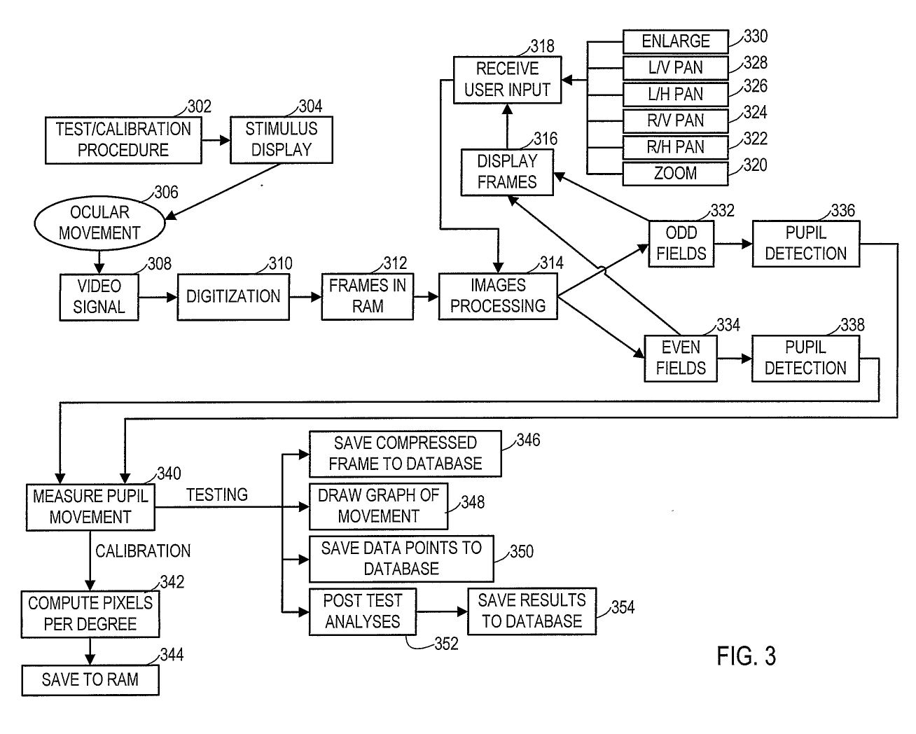

In order to initially obtain the ocular movement, the computer 108 utilizes a port of I/O system 206, such as a universal serial port, standard serial port, IEEE 1394 port, and the like to receive the incoming video signal(s) from one or more cameras 220, such as cameras of goggles 104 or cameras mounted to tri-pods or otherwise in a fixed position and focused on the subject 104. As discussed above, in certain embodiments the video signal(s) may already be digitized and even compressed prior to being received through a port of I/O system 206. In other embodiments, the video signal(s) may be analog such that a function of the I/O system 206 is to digitize the video signal(s). Further discussion of receiving the video signal is provided in relation to FIG. 3.

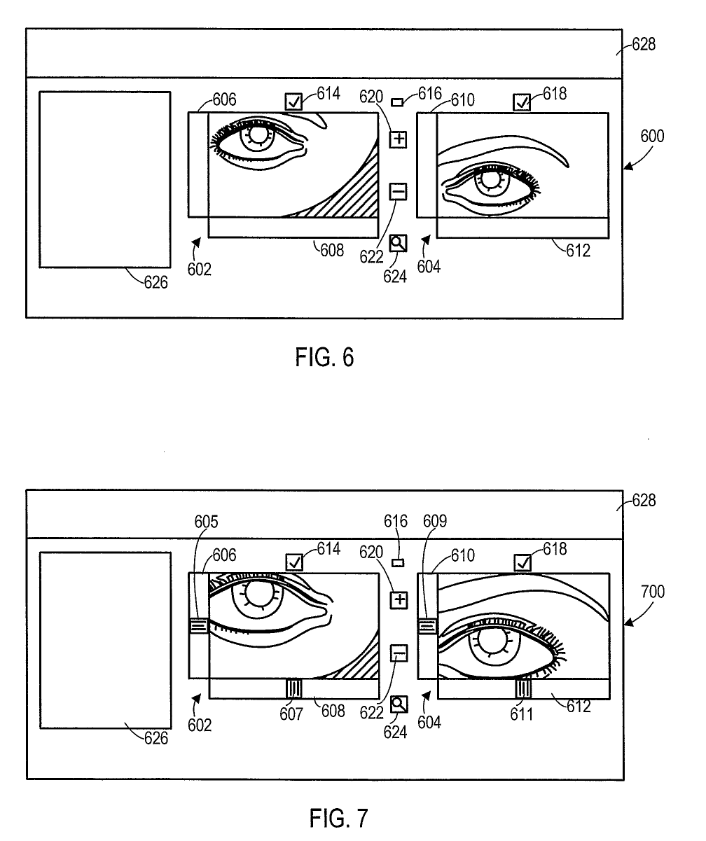

The computer system 108 of FIG. 2 also includes user interface devices (UID) 218 that allow a technician or clinician to interact with the computer, namely, the testing application 214 being implemented by the computer 108. The UID 218 may include a keyboard, mouse, touchscreen, voice command input, and the like. The testing application 214 is responsive to the user input when displaying the ocular movement in order to manipulate the display. The testing application itself may display graphical user interface controls, examples of which are shown below in relation to FIGS. 6-12, in order to receive user input via the mouse, touch screen, or other similar input device.

To generate the display of the ocular movement, the computer system 108 utilizes a display adapter 208 to generate display signals that are sent to a display monitor 112. Examples of such display signals include video graphics adapter (VGA) signals and the various advanced forms of that standard, such as super VGA, extended VGA, and so on. Additionally, to generate the stimulus if one is provided, the computer system 108 utilizes a display adapter 222 to generate display signals that are sent to a display monitor or projector 110.

FIG. 3 shows the various modules and operations involved in providing the display of ocular movement and in providing additional features of the testing application. At procedure operation 302, the clinician selects whether to begin a calibration or testing procedure for a subject. The calibration may be used in order to computer how many video frame pixels equate to a single degree of movement of the eyes of the subject. This calibration may be done where the movement of the eyes is to be measured, graphed, and analyzed by the testing application but is otherwise unnecessary for embodiments of displaying the ocular movement. Either the calibration or the testing procedure triggers a stimulus to be produced that causes the eyes of the subject to move, either voluntarily or involuntarily depending upon the test that is chosen.

The stimulus is displayed at display operation 304. At state 306, the ocular movement occurs as the eyes of the subject attempt respond to the stimulus being displayed. Video signals 308 are generated by the cameras where the video signals are a sequence of video frames, each frame providing an image of at least one eye of the subject so that the sequence of video frames shows the ocular movement. At digitization operation 310, each incoming video frame is digitized, and then at memory operation 312, the digitized video frame is loaded into memory.

In one embodiment where the video source is an NTSC video source, the frames arrive as individual fields, an odd field and an even field. Each field contains 480 interlaced lines, i.e., every other line contains information where the odd lines contain information for the odd field and the even lines contain information for the even field. The fields are receives every 1/60^{th} of a second so that a new frame is arriving every 1/30^{th} of a second. At image processing operation 314 of this particular embodiment, the odd and even fields are de-interlaced, such as by interpolation, to produce an odd field 332 and an even field 334. As the odd field 332 and even field 334 have been de-interlaced, they are each full frames occurring every 1/60^{th} of a second.

It will be appreciated that other non-NTSC video sources are also possible in other embodiments and in that case, the frames may be non-interlaced frames occurring every 1/60^{th} of a second such that de-interlacing is not needed to produce 60 full frames per second. It will also be appreciated that in alternative embodiments, the odd field and even field of an interlaced frame may be combined to produce a full frame that refreshes 30 times per second.

The image processing operation 314 may perform various operations upon the de-interlaced odd field 332 and even field 334 of this embodiment. For example, a histogram stretch of the image intensity may be performed to improve the contrast of the frames. The intensity range of the original image may not span the entire available range, and the histogram stretch spreads the intensities through the entire range.

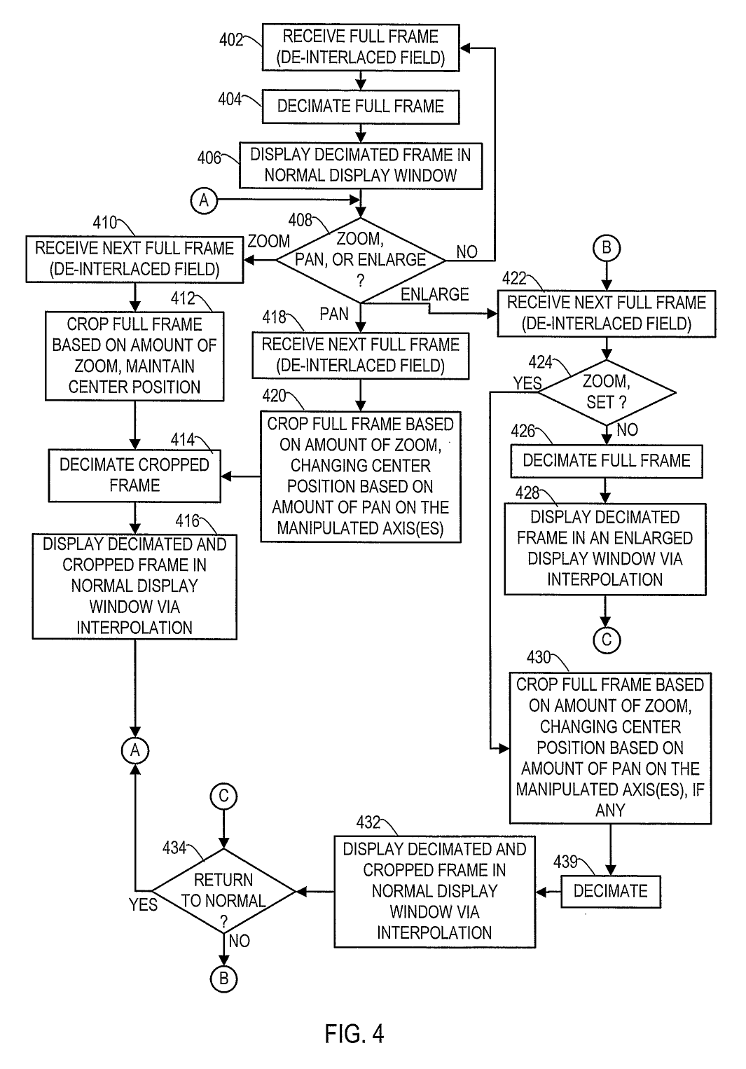

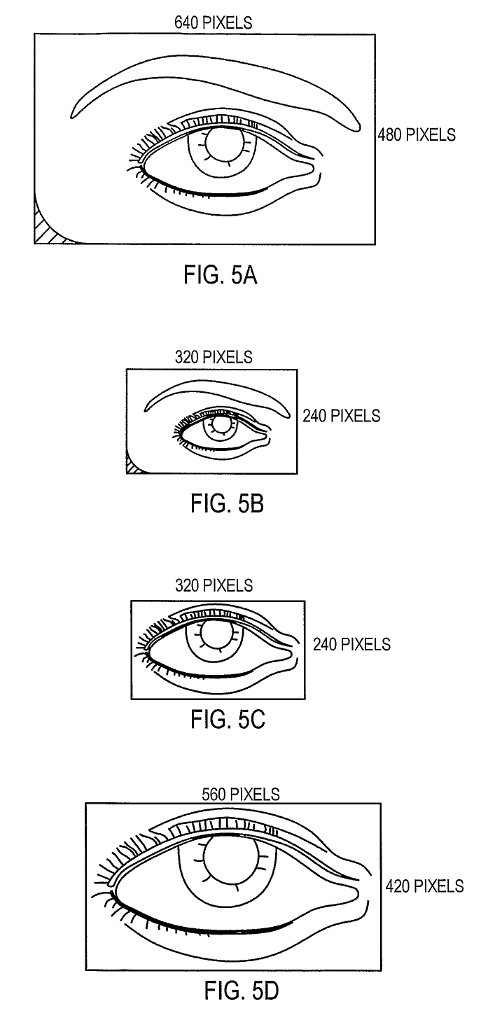

The image processing operation 314 may also perform operations to reduce the amount of data being handled. For an NTSC signal, the digitization and subsequent de-interlacing may result in a 640 pixel by 480 pixel frame. However, a lesser image may be desirable in order to reduce the amount of storage needed, especially considering that a separate video stream may be provided for each eye. So, the image processing operation 314 may decimate each frame to 320 pixels by 240 pixels. Additionally, only a portion of frame may be desired for display such that the frame is cropped, either before or after decimation. Further discussion of decimation and cropping is provided below in relation to FIGS. 4 and 5A-5D.

At this point, the de-interlaced fields that serve as frames can be displayed at display operation 316. Here the frames are displayed in sequence on the display screen. As discussed below in relation to FIGS. 5A-5D, the display resolution may be different than the original resolution of the digitized frame and may even be different than the resolution of the decimated frame. Interpolation may be used to display a frame having a resolution less than that of a display window in order to fill the display window with the frame. Operating systems such as the Windows® operating system by Microsoft Corp. of Redmond, Wash. provide display functions that take one image size and fill a display window of any given resolution by stretching the image along either or both axes via interpolation. Thus, the testing application may make use of the display functions of the underlying operating system. Alternatively, the testing application may implement a built-in interpolation to provide a frame that fills the display window.

During the display of the frames, user input may be received to allow the clinician to manipulate the display of the ocular movement at input operation 318. In one embodiment, the manipulation of the ocular movement may be a zoom input 320, a right eye horizontal pan input 322, a right eye vertical pan input 324, a left eye horizontal pan input 326, a left eye vertical pan input 328, or an enlarge input 330. The user input may take the form of selecting a control displayed in a graphical user interface, such as a control button or scroll bar, via a mouse click or touchscreen selection, or may take the form of one or a combination of keystrokes on a keyboard or a similar user initiated action.

In addition to these controls on the contents of the display window, timing controls may also be provided for purposes of receiving user input. For example, a stop or pause button may freeze the display with the current frame and re-start the sequence from the current frame. A time scale slider may be presented to allow the viewer to move the slider around on the scale to jump the video forward or backward in time. Each video frame has a time associated with it such that the time corresponding to the position of the slider points to a particular frame. That frame can be obtained from memory or mass storage and displayed to begin the sequence of frames from that point.

As discussed above, the testing application may provide additional features beyond displaying the ocular movement. Upon the fields 332, 334 being obtained, these fields may be analyzed to detect the location of the pupil within the frame at detection operations 336 and 338 and the change in location of the pupil from one frame to the next can be measured at measurement operation 340.

When the testing application is performing calibration, the measured pupil movement in terms of pixels can be used to compute the number of pixels per degree of ocular movement at computation operation 342. This pixels-per-degree constant can then be stored in memory at save operation 344 for subsequent use in graphing and analysis of the ocular movements.

When the testing application is performing an ocular movement test, the measured pupil movement can then be used to graph the movement at graph operation 348, with each of the data points being saved from memory to the database in mass storage. Post test analyses may be performed at analysis operation 352, such as determining whether the velocity of the ocular movement is within a normal range, and the results of this analysis may be saved to the database at save operation 354.

Additionally, the sequence of video frames may be compressed and saved to the database in relation to the measured points and results of the analyses. For example, the sequence of video frames may be compressed using a Haar wavelet transformation in order to save storage space and to make the database information more easily transported.

FIG. 4 shows one example of a set of logical operations performed by the testing application to perform the sequence of image processing, image display, and user input operations of FIG. 3. As discussed below, the clinician may zoom in on the image to remove artifacts that are otherwise present within the display window, such as the nosepiece of the goggles, to allow for easier viewing of the ocular movement and to aid in other features of the testing application, such as the pupil tracking where artifacts in the frame may cause problems. Furthermore, zooming provides the ability to pan within the frame so that the eye may be centered for better viewing and to aid in the other features so that physical adjustment of the goggles is unnecessary to properly center the eye. Additionally, the display window and frame within it may be enlarged to facilitate viewing from a distance.

In this illustrative embodiment shown in FIG. 4, the testing application receives the full frame, such as one of the de-interlaced fields, at frame operation 402. FIG. 5A shows an example of such a full frame, where in this example, the full frame is 640 pixels by 480 pixels. The full frame is then decimated at decimate operation 404 to produce a smaller frame but covering the same boundaries as the initial full frame. FIG. 5B shows an example of such a full frame after decimation, where the 640 pixel by 480 pixel frame is now 320 pixel by 240 pixel but still covers the same boundaries so that the content is the same but with less image precision. The decimated frame is then displayed in a normal display window having a particular display resolution at display operation 406. For example, the normal display window may call for a display resolution of 320 pixels by 240 pixels to fill the window such that the decimated frame of FIG. 5B fills the display window without interpolation.

At query operation 408, it is detected whether user input has been received to zoom, pan, or enlarge the frames being displayed. If there has yet to be a zoom, then there is no pan function available since the whole frame is being displayed. Upon the user selecting to zoom in on the full frame by some amount, the next full frame is then received at frame operation 410. Then, the full frame is cropped based on the amount of zoom that has been requested via the user input at crop operation 412. The center position of the frame is maintained as the center position of the resulting frame once it has been cropped since this is the first zoom attempt and no pan has been applied.

After cropping, which results in a frame that is less than 640 pixels by 480 pixels and that has boundaries moved inward, the resulting frame is then decimated at decimation operation 414. The cropped and decimated frame is now less than 320 pixels by 240 pixels. However, the cropped and decimated frame is now displayed in the normal display window of 320 pixels by 240 pixels by using interpolation to fill the window at display operation 416. FIG. 5C shows an example of a cropped and decimated frame that has been expanded to 320 pixels by 240 pixels via interpolation in order to fill the display window.

After having displayed the cropped and decimated frame, the process of cropping and decimating repeats for all subsequent frames being displayed until the clinician alters the zoom setting, pan setting, or requests and enlargement. It should be noted that the process of cropping and decimating may apply to both a sequence of video frames being received for the right eye as well as the sequence of video frames being received for the left eye. The zoom option may be presented to apply to both the right eye video and the left eye video, or to apply to one or the other at the option of the clinician.

Upon query operation 408 detecting that the clinician has requested to pan one of the ocular movement video displays, then the next full frame is received at frame operation 418. Then, the full frame is cropped in accordance with the amount of zoom that has been previously set. However, in performing the cropping, the center position is not maintained for the cropped frame relative to the original frame. Instead, the center position is moved based on the amount of horizontal or vertical panning that has been input by the clinician. After cropping based on the amount of zoom and pan that has been input thus far, then the cropped frame is decimated at decimation operation 414 and the cropped and decimated frame is displayed at display operation 416.

Again, after having displayed the cropped and decimated frame, the process of cropping based on zoom and pan and decimating repeats for all subsequent frames being displayed until the clinician alters the zoom setting, pan setting, or requests and enlargement. Upon query operation 408 detecting that the clinician has requested an enlargement of the display window and hence the frame being displayed, the next full frame is then received at frame operation 422. Query operation 424 detects whether a zoom has been set. If so, then the zoom can be preserved for the enlargement and the full frame is cropped based on the zoom, with the center position being changed for the cropped frame based on the amount of panning that has been set thus far at crop operation 430. The cropped frame is then decimated at decimation operation 434 and then the cropped and decimated frame is displayed in an enlarged display window via interpolation at display operation 432. An enlarged frame is shown in FIG. 5D, where the frame has been enlarged from a resolution of less than 320 pixels by 240 pixels to a display resolution of 560 pixels by 420 pixels via interpolation.

If the zoom has not been set, then the full frame is decimated at decimation operation 426 and then the decimated frame is displayed in an enlarged display window via interpolation at display operation 428. After the image is displayed, either as a cropped and decimated frame at display operation 432 or as a decimated frame at display operation 428, then query operation 434 detects whether the clinician has selected to return the display window to the normal resolution. If not, then the process repeats for the subsequent frames to crop when necessary based on zoom and pan, decimate, and display in the enlarged display window. Once the clinician has selected to return the display of the frame sequence to the normal size window, then operational flow returns to query operation 408 where it is again detected whether the clinician has provided input to alter the zoom, pan, or enlargement of the frames being displayed.

FIG. 6 shows an example of a screenshot 600 from a testing application where two video signals of ocular movement are being displayed, one video signal for a right eye of a subject and one video signal for a left eye of the subject. The screenshot provides two normal sized display windows, a first display window 602 showing the right eye of the subject and a second display window 604 showing the left eye of the subject. This screenshot shows full frames as they are initially displayed prior to receiving any zoom, pan, or enlargement request by the clinician. As can be seen, the eyes of the subject are not centered within the display windows and are not aligned relative to one another so that it would be difficult for a clinician to watch the ocular movement of the two eyes. Furthermore, artifacts are present within the displayed frames, namely a nosepiece of goggles being worn by the subject and being used to capture the video signals.

Rather than physically adjusting and re-positioning the goggles on the face of the subject in an attempt to properly center and align the eyes within the display windows, the clinician utilizes video frame manipulation controls, such as controls provided in the graphical user interface of the display. The manipulation controls of this particular example include vertical scrollbars 606 and 610 as well as horizontal scrollbars 608 and 612 that may be used to pan the frames vertically and horizontally to thereby control what portions of the frames are displayed within the window. However, these scrollbars are not active within this screenshot because the full frame is being displayed as no zoom input has yet been received.

In order to zoom in on the frames being displayed, zoom controls are provided. A zoom in button 620 allows the clinician to click the button and zoom in by a set amount per click. Likewise, a zoom out button 622 allows the clinician to click the button and zoom out by a set amount per click. The zoom in is achieved in this example by cropping the frame, either before or after decimating, and then displaying the cropped and decimated frame in the display window via interpolation. The amount of cropping per click, and hence the amount of zoom to be achieved per click, or per unit of time (e.g., 0.5 seconds) that the zoom button is being pressed, is a matter of design choice but one example is a reduction of 5% of the pixels per click or per unit of time pressed. Rather than having a single button to click zoom in and another single button to click to zoom back out, it will be appreciated that other manners of receiving a zoom in or zoom out are possible, such as by presenting a range of percentages of zoom, either numerically or as a scale, and receiving a selection of that percentage.

The zoom in button 620 and zoom out button 622 may be set to work with only a single display window, and therefore a single eye, or with both windows and both eyes. A set of checkboxes or other manner of receiving a user selection may be presented for this purpose. As shown, a right eye zoom checkbox 614, a left eye zoom checkbox 618, and an independent eye zoom checkbox 616 are presented, and the clinician may check or uncheck these boxes to control which windows are zoomed. Clicking the independent eye zoom 616 unchecks the checkboxes 614 and 618 and allows the clinician to then check either box to re-establish zoom for that corresponding display window. Clicking the independent eye zoom 616 again re-establishes zoom for both display windows. FIG. 7, discussed below, shows the result of zooming in.

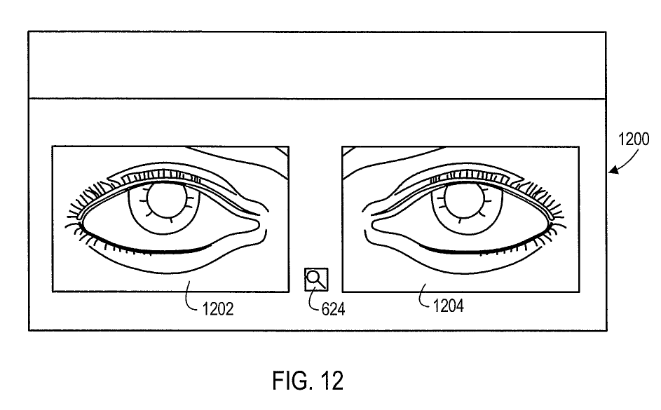

In addition to providing the zoom and pan options, an enlarge button 624 may be provided. The clinician may wish to enlarge the display windows, and hence the size of the eyes being displayed such as if the clinician plans to step away from the display screen but wishes to continue viewing the ocular movement from a distance. The result of using the enlargement option is discussed below in relation to FIG. 12.

The graphical user interface of the screenshot 600 may include additional sections beyond the video display windows 602, 604. For example, a dialog box 626 may be presented that lists the different tests that have been performed or that are to be performed along with an identification of the current subject. Furthermore, a menu bar 628 may be presented to allow the clinician to select various testing options, such as the particular type of test to perform.

Once the clinician selects the zoom in button 620, assuming the zoom is set to work with both display windows, the size of the objects in the frame are enlarged but less of the frame is shown in the display window as illustrated in the screenshot 700 of FIG. 7. After zooming, it can be seen that the center position has been maintained and the content of the display windows has grow in size. However, it can further be seen that the eyes are still not centered nor aligned with one another.

Now that the zoom has occurred, the pan controls become functional since there is more of the frame than what is being displayed in the display windows 602, 604. The scrollbar 606 now has a slider 605, the scrollbar 608 now has a slider 607, the scrollbar 610 now has a slider 609, and the scrollbar 612 now has a slider 611. The clinician can click and hold on one of these sliders and then move the slider within its corresponding scrollbar to result in a corresponding change to the portion of the frame being displayed. For example, the movement of slider 605 upward causes the center of the cropping to be shifted downward so that content toward to the bottom of the full frame becomes visible in the display while content toward the top of the full frame is cropped out.

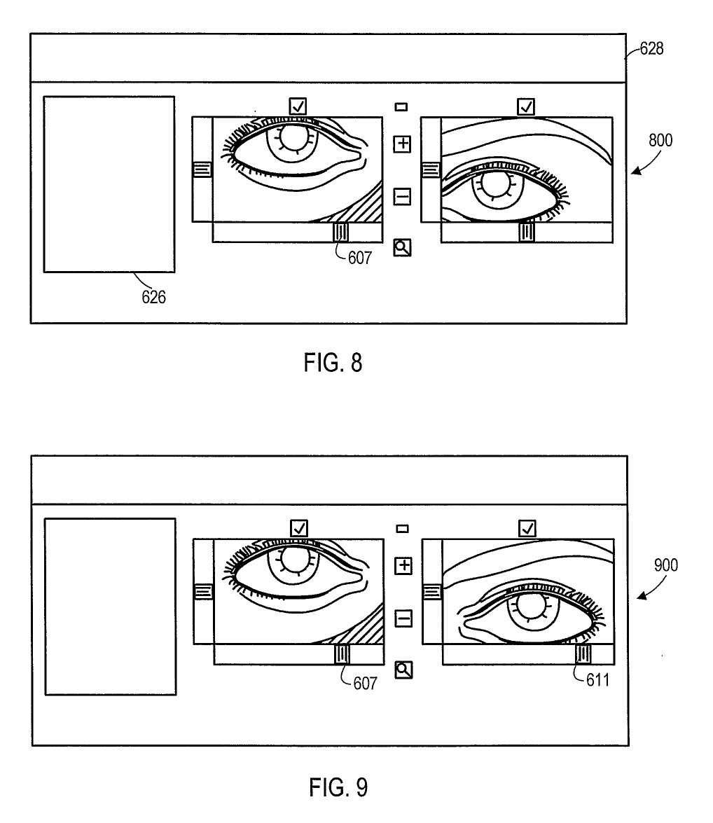

FIG. 8 shows a screenshot 800 after the clinician has moved the slider 607 to the right to thereby shift the center of the cropping to the left. This has the effect of moving the right eye of the subject (the eye of the left display window) to the right, and since the right eye was to the left of center, the movement of the slider 607 to the right has moved the right eye closer to horizontal center. The artifacts, namely the nosepiece of the goggles, are now almost eliminated from the frame.

FIG. 9 shows a screenshot 900 after the clinician has moved the slider 611 to the right to thereby shift the center of the cropping to the left. This has the effect of moving the left eye of the subject (the eye of the right display window) to the right, and since the left eye was to the left of center, the movement of the slider 611 to the right has moved the left eye closer to horizontal center.

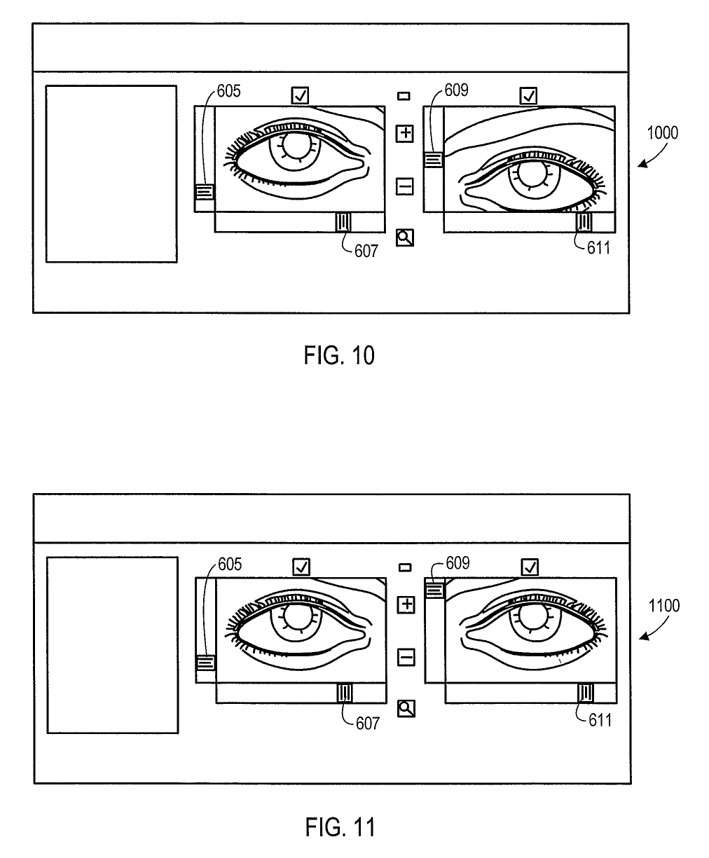

FIG. 10 shows a screenshot 1000 after the clinician has moved the slider 605 to downward to thereby shift the center of the cropping upward. This has the effect of moving the right eye downward, and since the right eye was above center, the movement of the slider 605 downward has moved the right eye closer to vertical center. The artifacts, namely the nosepiece of the goggles, are now completely eliminated from the frame.

FIG. 11 shows a screenshot 1100 after the clinician has moved the slider 609 to upward to thereby shift the center of the cropping downward. This has the effect of moving the left eye upward, and since the left eye was below center, the movement of the slider 605 upward has moved the left eye closer to vertical center. As can be seen in FIG. 11, the eyes of each display window 602, 604 are now substantially centered in the horizontal and vertical axes and are substantially aligned with the opposite eye. The clinician now has a good view of both eyes and can relate movement of one eye relative to the other. This has been accomplished without physically adjusting or re-positioning the goggles on the patient.

FIG. 12 shows a screenshot 1200 after the clinician has decided to enlarge the eyes by selecting the enlarge button 624. In the example shown, the clinician has chosen to enlarge the frames after having zoomed in and panned to center and align the eyes. It will be appreciated that the clinician may utilize the enlarge option prior to zooming or if after zooming, prior to panning. As the display windows 1202 and 1204 are now larger than the display windows 602 and 604, the clinician can step away from the screen but still adequately view the ocular movement. Should the clinician wish to return to a normal display window size, the clinician can select the enlarge button 624 once more. As shown, the zooming and panning features are not provided while the video display windows are enlarged. However, it will be appreciated that in other embodiments, the zoom in, zoom out, and panning features may also be provided while the video display windows are enlarged.

While the invention has been particularly shown and described with reference to various embodiments thereof, it will be understood by those skilled in the art that various other changes in the form and details may be made therein without departing from the spirit and scope of the invention.

1. A method of providing a display of ocular movement, comprising:
obtaining a sequence of digitized video frames of the ocular movement at a first resolution;
displaying in sequence a portion of each frame of the sequence of digitized video frames of the ocular movement, the portion being at a second resolution lower than the first resolution and being displayed at a first display resolution;
receiving a first user input while displaying in sequence the portion of each frame; and
in response to the received first user input, panning the portion within the subsequent frames of the ocular movement.

2. The method of claim 1, further comprising displaying a first image scroll control alongside a first axis of the display of the portion of each frame of the sequence, and wherein receiving the first user input comprises receiving manipulation of the first image scroll control to pan the portion by changing the portion along the first axis.

3. The method of claim 2, further comprising displaying a second image scroll control alongside a second axis of the display of the portion of each frame of the sequence, and wherein receiving the first user input further comprises receiving manipulation of the second image scroll control to pan the portion by changing the portion along the second axis.

4. The method of claim 1, further comprising:
receiving a second user input while displaying in sequence the portion; and
in response to the received second user input, changing the resolution of the portion of each frame of the sequence being displayed and displaying the portion having the changed resolution of each frame at the first display resolution.

5. The method of claim 1, wherein the first resolution is 640 pixels by 480 pixels, the method further comprising cropping the frame to a resolution less than 640 pixels by 480 pixels and decimating the cropped frame to produce the portion being displayed, wherein the portion is less than 320 pixels by 240 pixels.

6. The method of claim 5, further comprising interpolating within the portion to display the portion at the first display resolution, wherein the first display resolution is 320 pixels by 240 pixels.

7. The method of claim 1, further comprising:
receiving a third user input while displaying in sequence the portion; and
in response to the received third user input, displaying the portion of each frame of the sequence at a second display resolution that is larger than the first display resolution.

8. The method of claim 7, further comprising interpolating within the portion to display the portion at the second display resolution, wherein the second display resolution is 560 pixels by 420 pixels.

9. A computer system for displaying ocular movement, comprising:
a first input receiving a sequence of digitized video frames of the ocular movement at a first resolution;
a memory storing at least a portion of each digitized video frame being received;
a second input receiving a first user input; and
a processor that initiates displaying in sequence a portion of each frame of the sequence of digitized video frames of the ocular movement, the portion being at a second resolution lower than the first resolution and being displayed at a first display resolution, and in response to the received first user input initiates panning the portion within the subsequent frames of the ocular movement being displayed.

10. The computer system of claim 9, wherein the processor initiates displaying a first image scroll control alongside a first axis of the display of the portion of each frame of the sequence, and wherein the second input receives the first user input as manipulation of the first image scroll control to pan the portion by changing the portion along the first axis.

11. The computer system of claim 10, wherein the processor initiates displaying a second image scroll control alongside a second axis of the display of the portion of each frame of the sequence, and wherein the second input receives the first user input as manipulation of the second image scroll control to pan the portion by changing the portion along the second axis.

12. The computer system of claim 9, wherein the second input receives a second user input while the processor initiates displaying in sequence the portion, and in response to the received second user input the processor changes the resolution of the portion of each frame of the sequence being displayed and initiates displaying the portion having the changed resolution at the first display resolution.

13. The computer system of claim 9, wherein the first resolution is 640 pixels by 480 pixels, and wherein the processor crops the frame to a resolution less than 640 pixels by 480 pixels and decimates the cropped frame to produce the portion being displayed, wherein the portion is less than 320 pixels by 240 pixels.

14. The computer system of claim 13, further comprising interpolating within the portion to display the portion at the first display resolution, wherein the first display resolution is 320 pixels by 240 pixels.

15. The computer system of claim 9, wherein the second input receives a third user input while the processor initiates displaying in sequence the portion, and in response to the received third user input the processor initiates displaying the portion of each frame of the sequence at a second display resolution that is larger than the first display resolution.

16. The computer system of claim 15, further comprising interpolating within the portion to display the portion at the second display resolution, wherein the second display resolution is 560 pixels by 420 pixels.

17. A computer readable medium having instructions encoded thereon that perform acts comprising:
obtaining a sequence of digitized video frames of the ocular movement at a first resolution;
displaying in sequence a portion of each frame of the sequence of digitized video frames of the ocular movement, the portion being at a second resolution lower than the first resolution and being displayed at a first display resolution;
receiving a first user input while displaying in sequence the portion of each frame; and
in response to the received first user input, panning the portion within the subsequent frames of the ocular movement.

18. The computer readable medium of claim 17, wherein the acts further comprise displaying a first image scroll control alongside a first axis of the display of the portion of each frame of the sequence, and wherein receiving the first user input comprises receiving manipulation of the first image scroll control to pan the portion by changing the portion along the first axis.

19. The computer readable medium of claim 18, wherein the acts further comprise displaying a second image scroll control alongside a second axis of the display of the portion of each frame of the sequence, and wherein receiving the first user input further comprises receiving manipulation of the second image scroll control to pan the portion by changing the portion along the second axis.

20. The computer readable medium of claim 17, wherein the acts further comprise: receiving a second user input while displaying in sequence the portion; and
in response to the received second user input, changing the resolution of the portion of each frame of the sequence being displayed and displaying the portion having the changed resolution of each frame at the first display resolution.

21. The computer readable medium of claim 17, wherein the first resolution is 640 pixels by 480 pixels, the acts further comprising cropping the frame to a resolution less than 640 pixels by 480 pixels and decimating the cropped frame to produce the portion being displayed, wherein the portion is less than 320 pixels by 240 pixels.

22. The computer readable medium of claim 21, wherein the acts further comprise interpolating within the portion to display the portion at the first display resolution, wherein the first display resolution is 320 pixels by 240 pixels.

23. The computer readable medium of claim 1, wherein the acts further comprise:
receiving a third user input while displaying in sequence the portion; and
in response to the received third user input, displaying the portion of each frame of the sequence at a second display resolution that is larger than the first display resolution.

24. The computer readable medium of claim 23, wherein the acts further comprise interpolating within the portion to display the portion at the second display resolution, wherein the second display resolution is 560 pixels by 420 pixels.
